(19) United States
(12) Patent Application Publication
(10) Pub. No.: US 2019/0275261 A1
(43) Pub. Date: Sep. 12, 2019

(54) SAFETY LOCKED NEEDLE

(71) Applicant: SPIRATION, INC., d/b/a OLYMPUS RESPIRATORY AMERICA, Redmond, WA (US)

(72) Inventors: Matt Nickeson, Redmond, WA (US); Christine Jurevicius, Redmond, WA (US)

(21) Appl. No.: 16/461,859

(22) PCT Filed: Jan. 12, 2017
(86) PCT No.: PCT/US2017/013107

§ 371 Date: May 17, 2019

(51) Int. Cl. A61M 5/32; A61M 5/50; A61M 5/31; A61M 5/315

(52) U.S. Cl. CPC A61M 5/3234; A61M 5/508; A61M 5/502; A61M 5/31505; A61M 5/3135

(57) ABSTRACT

The present teachings relate generally to a medical sampling device assembly (10), comprising: a sampling portion (20) disposed at a distal end (12) of the assembly; handle portion (30) disposed at a proximal end (14) of the assembly and including a bias member (32), a locking member (34) coupled to the bias member, and an indicator member (36); an intermediate portion (40) disposed between and interconnecting the sampling portion and the handle portion; and a sampling member (50) slidably disposed within the handle portion, the intermediate portion, and the sampling portion and protruding distally thereof, and in mechanical communication with the bias member; wherein the indicator member is deployed and the sampling member retracts permanently and completely into the sampling portion upon decoupling the locking member from the sampling member.

### FIELD

The present teachings relate to a medical sampling device assembly with improved safety features, more particularly a single use medical sampling device assembly with a remote retraction mechanism and visual retraction indicator.

### BACKGROUND

There is an ongoing need in the healthcare industry to provide safe sampling devices. It may be desirable to have sampling devices that are configured to be single use. It may also be desirable to have a device, that once used, locks in a position that prevents inadvertent needle jabs. It may also be desirable to have one or more visual indicators that indicate to a user whether the device has been used or not, or if the needle is deployed or retracted.

While many existing materials and components meet some of the above needs, there continues to be a need for improved single use medical sampling device assembly.

### SUMMARY

The present teachings relate generally to a single use medical sampling device assembly with a remote retraction mechanism and visual retraction indicator. The device includes at least a handle, a sampling portion, a sampling member, an indicator portion, and a bias member for retracting the sampling member into the sampling portion.

### DESCRIPTION OF THE DRAWINGS

FIG. 1 is a sectional view of one example of the present invention with the sampling member deployed.

FIG. 2 is a sectional view of one example of the present invention with the sampling member retracted.

FIG. 3 is a sectional view of another example of the present invention with the sampling member deployed.

FIG. 4 is a sectional view of another example of the present invention with the sampling member retracted.

### DETAILED DESCRIPTION

The explanations and illustrations presented herein are intended to acquaint others skilled in the art with the invention, its principles, and its practical application. The specific embodiments of the present invention as set forth are not intended as being exhaustive or limiting of the invention. The scope of the invention should be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description. The present teachings relate generally to a medical sampling device assembly 10, comprising: a sampling portion a sampling portion 20 disposed at a distal end 12 of the assembly; a handle portion 30 disposed at a proximal end 14 of the assembly and including a bias member 32, a locking member 34 mechanically coupled to the bias member, and an indicator member 36; an intermediate portion 40 disposed between and interconnecting the sampling portion and the handle portion; and a sampling member 50 slidably disposed within the handle portion, the intermediate portion, and the sampling portion and protruding distally thereof, and in mechanical communication with the bias member; wherein the indicator member is deployed and the sampling member retracts permanently and completely into the sampling portion upon decoupling the locking member from the device.

It is also contemplated that present invention may include that the sampling member includes wherein the indicator member is affixed to the slidable stop block 60; indicator member is affixed to the sampling member; and the slidabe stop block forms a portion of the locking mechanism. Each physical element is described in more detail below.

Medical Sampling Device Assembly 10

It is contemplated that a new and inventive sampling device for use in the medical field is disclosed herein. It is contemplated that the assembly includes a mechanism or feature that can be incorporated into needles or other sampling devices that will function to place them into a safely retracted mode when activated. This should not interfere with the normal use of the device until activated. As described below, this mechanism or feature could use a bias member or locking member to keep a device in an extended state, and removing or decoupling the locking member would cause the bias member to permanently retract the device. A visual flag or indicator member could also ‘pop out’ to inform the user that the device is safe/disabled. The present invention may provide multiple ways for a user to observe the safety of the device, for example, the locking member (e.g. pin) is moved or missing or the indicator member or flag is deployed or activated. Each component of the assembly is described in further detail below.

Sampling Portion 20/Sampling Member 50

It is contemplated that the sampling device 10 includes a sampling portion. The sampling portion generally is connected to and extends distally from a handle (via the intermediate portion 40) and functions as the portion of the device that is inserted into a body. The sampling portion may include a hollow shaft portion or lumen that is disposed centrally within the sampling portion. Within the hollow shaft portion, it is contemplated that there is a sidably disposed/movable sample member. The sample member may include a sharp tip at the distal end (e.g. needle or other sampling means). The sample member may be a fully hollow, partially hollow, or solid tube like structure. It is contemplated that the sample member may be ridged or flexible along portions of it.

Handle Portion 30

It is contemplated that the sampling device 10 includes a handle portion. The handle portion is generally disposed at the proximal end 14 of the device 10. It may function as a hand grip for a user when using the device. It additionally may house or be part of the mechanisms that provide for the safety features of the present invention. In one preferred embodiment, the housing included a slidable block, a bias member, a locking member (at least a portion of one) disposed therein.

Bias Member 32

It is contemplated that the sampling device 10 includes a bias member. The bias member functions to provide the force required to retract the sample member when the locking member is activated. In one preferred embodiment, the bias member is a coil spring disposed about part of the sampling member and expands when locking member is activated causing the sample member to retract into the sampling portion. In another preferred embodiment, the bias member is a coil spring disposed about part of the sampling member and contracts when locking member is activated causing the sample member to retract into the sampling portion. In yet another preferred embodiment, the bias member is a coil spring disposed adjacent to the sampling member and contracts when locking member is activated causing the sample member to retract into the sampling portion.

Locking Member 34

It is contemplated that the sampling device 10 includes a locking member. The locking member functions to secure the sampling member in a deployed position (e.g. protruding distally of the sample portion) and upon activation, to allow for the bias member to retract it permanently into the sampling member. In one preferred embodiment, the locking member is comprised of a removable pin that fits into a detent or notch in the slidable stop block or at least mechanically connected to the block or the sampling member. It is contemplated in the preferred embodiment, that the user would actuate the locking member by partially or totally removing the pin from the housing, thus allowing the bias member to move (e.g. expand or contract in the case of a spring) and providing a visual indication that the sampling device was in a safely retracted mode.

Indicator Member 36

It is contemplated that the sampling device 10 includes an indicator member. The indicator member functions as a visual indicator that indicates when the sampling member is either in a deployed position or a retracted position. In one preferred embodiment, the indicator member is comprised of a shaft that is about even with or protrudes from the proximal end of the handle portion at a first shorter distance when the sampling member is deployed and at a second longer distance when the sampling member is retracted.

Slidable Stop Block

It is contemplated that the sampling device includes a slidable stop bock. The stop block may have one or more functions. It may function as an interface or reaction surface for the bias member. It may function as part of the locking member, for example as one side of a pin/detent locking design. It generally is contemplated to be slidably disposed within the handle portion and moves proximally when the bias member is allowed to move (e.g. locking member is decoupled).

### ILLUSTRATIVE EXAMPLES

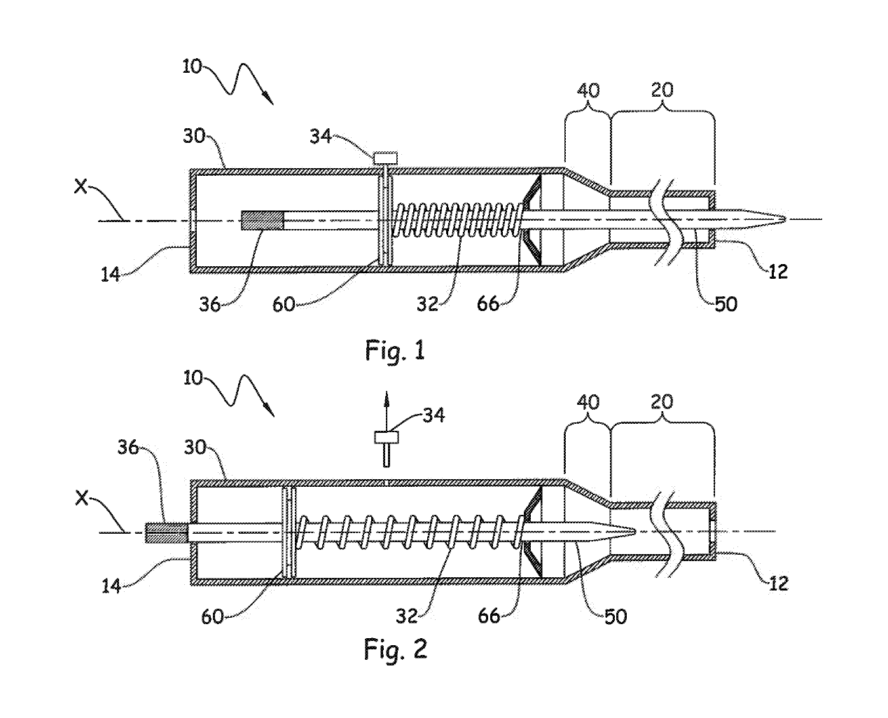

As shown in FIGS. 1 and 2, a medical sampling device 10 with an expanding bias member 32 is shown. FIG. 1 illustrates the device 10 with the sampling member 50 extended beyond the distal end 12 of the sampling portion 20. The locking member 34 is engaged to prevent the bias member 32 from expanding and causing the sample member 50 from retracting into device 10.

FIG. 2 illustrates the device 10 with the sampling member 50 retracted and disposed inside the distal end of the sampling portion 20. The locking member 34 has been disengaged and the bias member 32 has expanded, causing the sample member 50 to retract into device 10. Additionally, the indicator member 36 is now protruding for the proximal end 14 of the device, thus providing a visual indication that the sampling member 50 is disposed internally to the device 10. In this illustrative example, the expanding bias member 32 is disposed axially about the sampling member.

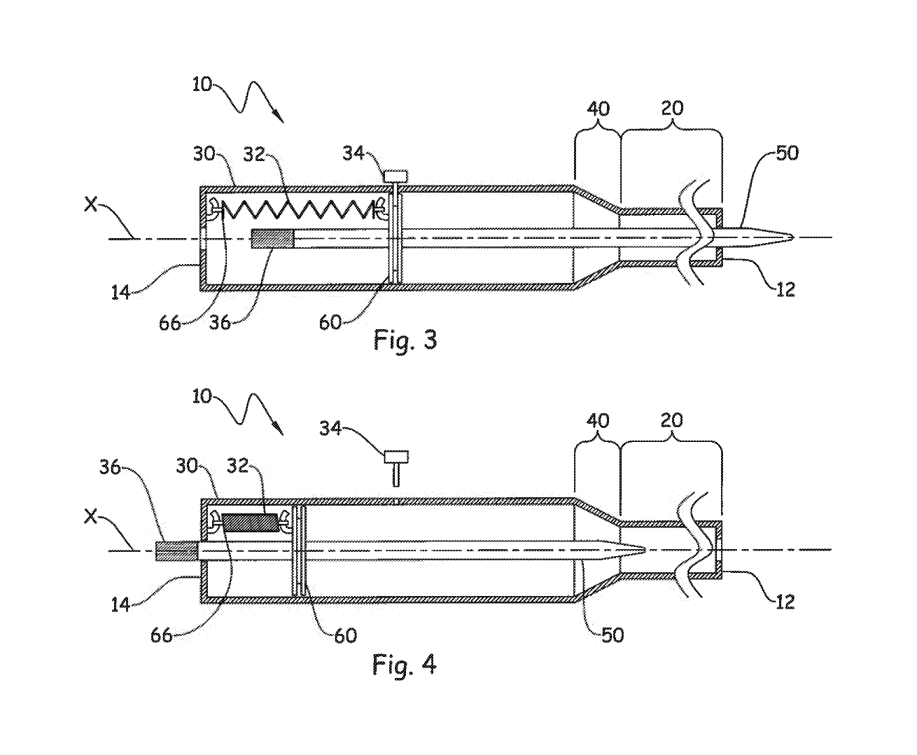

As shown in FIGS. 3 and 4, a medical sampling device 10 with a contracting bias member 32 is shown. FIG. 3 illustrates the device 10 with the sampling member 50 extended beyond the distal end 12 of the sampling portion 20. The locking member 34 is engaged to prevent the bias member 32 from contracting and causing the sample member 50 from retracting into device 10.

FIG. 4 illustrates the device 10 with the sampling member 50 retracted and disposed inside the distal end of the sampling portion 20. The locking member 34 has been disengaged and the bias member 32 has contracted, causing the sample member 50 to retract into device 10. Additionally, the indicator member 36 is now protruding for the proximal end 14 of the device, thus providing a visual indication that the sampling member 50 is disposed internally to the device 10. In this illustrative example, the contracting bias member 32 is disposed adjacent to the sampling member, but it is contemplated that it may be preferred to be disposed axially about the sampling member.

Unless otherwise stated, any test method standard referenced herein is for the version existing as of the earliest filing date in which the standard is recited.

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of, or even consist of the elements, ingredients, components or steps. Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps. Moreover, unless expressly set forth, the recitation of “first”, “second”, or the like does not preclude additional ingredients, steps, or other elements. All references herein to elements or metals belonging to a certain Group refer to the Periodic Table of the Elements published and copyrighted by CRC Press, Inc., 1989. Any reference to the Group or Groups shall be to the Group or Groups as reflected in this Periodic Table of the Elements using the IUPAC system for numbering groups. It is understood that the above description is intended to be illustrative and not restrictive.

Many embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

1. A medical sampling device assembly, comprising:
a. a sampling portion disposed at a distal end of the assembly;
b. a handle portion disposed at a proximal end of the assembly and including:
1. a bias member,
2. a locking member mechanically coupled to the bias member, and
3. an indicator member;
c. an intermediate portion disposed between and interconnecting the sampling portion and the handle portion; and
d. a sampling member slidably disposed within the handle portion, the intermediate portion, and the sampling portion and protruding distally thereof, and being in mechanical communication with the bias member;
wherein the indicator member is deployed and the sampling member retracts permanently and completely into the sampling portion upon decoupling the locking member.

2. The medical sampling device assembly according to claim 1:
wherein the medical sampling device includes a slidable stop block disposed therein and the bias member is disposed between the slidable stop block and a distal end of the handle portion

3. The medical sampling device assembly according to claim 1:
wherein the medical sampling device includes a slidable stop block disposed therein and the bias member is disposed between the slidable stop block and a proximal end of the handle portion.

4. The medical sampling device assembly according to claim 2:
wherein the indicator member is affixed to the slidable stop block.

5. The medical sampling device assembly according to claim 1:
wherein the indicator member is affixed to the sampling member.

6. The medical sampling device assembly according to claim 1:
wherein the slidabe stop block forms a portion of the locking mechanism.

7. The medical sampling device of claim 1, wherein the locking member is a pin that is totally removable from the handle portion.

8. The medical sampling device of claim 1, wherein the locking member extends through a wall of the handle portion and into the slidable stop block to prevent retraction of the sampling member.

9. The medical sampling device of claim 6, wherein the locking mechanism extends into the slidable stop block.

10. The mechanical sampling device of claim 9, wherein the slidable stop block includes a notch or detent that receives a portion of the locking mechanism.

11. The medical sampling device of claim 2, wherein the slidable stop block is connected to the sampling member.

12. The medical sampling device of claim 1, wherein the indicator member extends through the proximal end of the assembly when the indicator member is deployed.
